(19) United States
(12) Patent Application Publication
(10) Pub. No.: US 2022/0160710 A1
(43) Pub. Date: May 26, 2022

(54) COMPOSITIONS AND METHODS FOR TREATING OR PREVENTING ALZHEIMER'S DISEASE

(71) Applicant: University of Virginia Patent Foundation, Charlottesville, VA (US)

(72) Inventor: Jayakrishna Ambati, Charlottesville, VA (US)

(73) Assignee: University of Virginia Patent Foundation, Charlottesville, VA (US)

(21) Appl. No.: 17/436,882

(22) PCT Filed: Mar. 9, 2020
(86) PCT No.: PCT/US2020/021704

§ 371 Date: Sep. 7, 2021

Related U.S. Application Data
(60) Provisional application No. 62/825,330, filed on Mar. 28, 2019.
(60) Provisional application No. 62/815,629, filed on Mar. 8, 2019.

(51) Int. Cl. A61K 31/513; A61K 45/06; A61P 25/28

(52) U.S. Cl. CPC A61K 31/513; A61P 25/28; A61K 45/06

(57) ABSTRACT

Methods for treating and/or inhibiting progression of neurological diseases, conditions, and/or disorders. In some embodiments, the methods include administering to as subject in need thereof a composition that includes a reverse transcriptase inhibitor. Also provided are methods for inhibiting development of amyloid beta peptide (Aβ) in subjects, methods for inhibiting microglial cell death, and composition for use in the presently disclosed methods.

### CROSS REFERENCE TO RELATED APPLICATION

The presently disclosed subject matter claims the benefit of U.S. Provisional Patent Application Serial Nos. 62/815,629, filed Mar. 8, 2019, and 62/825,330, filed Mar. 28, 2019. The disclosure of each of these applications is incorporated herein by reference in its entirety.

### GOVERNMENT INTEREST

This invention was made with government support under Grant Nos. GM114862, EY028027, and EY029799 awarded by The National Institutes of Health. The government has certain rights in the invention.

### BACKGROUND

Various cellular and molecular processes contribute to the development of several neurological diseases. The incidences of neurological diseases will thus continue to rise as the population ages.

Alzheimer's disease (AD) is an example of such an age-related neurological disease. AD is characterized by memory loss, cognitive impairment, and behavioral changes. More than 15 million people suffer from AD worldwide and this disease is the 7th leading cause of death in the United States. Many neurological disorders can be attributed to deregulated protein levels in the brain. AD pathology is characterized by extra-cellular amyloid beta (Aβ) neuritic plaques and intracellular neurofibrillary tangles. Aβ plaques are toxic and progressively accumulate in the brain throughout the duration of the disease, resulting in neuronal loss and cortical atrophy. Excessive Aβ accumulation eventually involves much of the neocortex, hippocampus and many subcortical structures.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for treating and/or inhibiting progression of neurological diseases, conditions, and/or disorders in subjects suffering from and/or at risk for developing the neurological diseases, conditions, and/or disorders. In some embodiments, the methods comprise administering to a subject in need thereof a composition comprising a reverse transcriptase inhibitor in an amount and via a route effective for treating and/or inhibiting progression of the neurological disease, condition, and/or disorder in the subject. In some embodiments, the neurological disease, condition, and/or disorder is selected from the group consisting of Alzheimer's Disease (AD), cerebral amyloid angiopathy, cognitive impairment, mild cognitive impairment, Alzheimer's disease-related attention deficit symptoms, Alzheimer's disease-related neural degeneration, degenerative dementia, senile dementia, cerebral vascular dementia, alcoholic dementia, Parkinson's disease-related dementia, tic disorder, corticobasal ganglionic degeneration, and HIV-associated neurocognitive disorder (HAND), or comprises any combination thereof. In some embodiments, the composition comprises, consists essentially of, or consists of a reverse transcriptase inhibitor selected from the group consisting of a nucleoside reverse transcriptase inhibitor (NRTI), a non-nucleoside reverse transcriptase inhibitor (NNRTI), or any combination thereof. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the NNRTI is selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

In some embodiments, the presently disclosed subject matter further comprises administering to the subject an additional treatment, optionally an additional AD treatment. In some embodiments, the additional AD treatment is selected from the group consisting of treatment with an acetylcholinesterase (AChE) inhibitor, optionally donepezil, rivastigmine, and/or galantamine; treatment with an N-methyl-d-aspartate receptor (NMDAR) antagonist, optionally, memantine; treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126, miR-145, miR-195, miR-21, and miR-29b; and combinations thereof.

In some embodiments, the presently disclosed subject matter also relates to methods for inhibiting development of amyloid beta peptide (Aβ) in a subject suffering from Alzheimer's Disease (AD), the method comprising administering to the subject a composition comprising a reverse transcriptase inhibitor in an amount and via a route effective for treating and/or inhibiting progression of the AD in the subject. In some embodiments, the composition comprises, consists essentially of, or consists of a reverse transcriptase inhibitor selected from the group consisting of a nucleoside reverse transcriptase inhibitor (NRTI), a non-nucleoside reverse transcriptase inhibitor (NNRTI), or any combination thereof. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the NNRTI is selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

In some embodiments, the presently disclosed methods further comprise administering to the subject an additional treatment, optionally an additional AD treatment. In some embodiments, additional AD treatment is selected from the group consisting of treatment with an acetylcholinesterase (AChE) inhibitor, optionally donepezil, rivastigmine, and/or galantamine; treatment with an N-methyl-d-aspartate receptor (NMDAR) antagonist, optionally, memantine; treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126, miR-145, miR-195, miR-21, and miR-29b; and combinations thereof.

In some embodiments, the presently disclosed subject matter also relates to methods for inhibiting microglial cell death. In some embodiments, the presently disclosed methods comprise administering to a subject in need thereof a composition comprising a reverse transcriptase inhibitor in an amount and via a route effective for inhibiting microglial cell death in the subject. In some embodiments, the composition comprises, consists essentially of, or consists of a reverse transcriptase inhibitor selected from the group consisting of a nucleoside reverse transcriptase inhibitor (NRTI), a non-nucleoside reverse transcriptase inhibitor (NNRTI), or any combination thereof. In some embodiments, NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the NNRTI is selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

In some embodiments of the presently disclosed methods further comprise administering to the subject an additional treatment, which in some embodiments is selected from the group consisting of treatment with an acetylcholinesterase (AChE) inhibitor, optionally donepezil, rivastigmine, and/or galantamine; treatment with an N-methyl-d-aspartate receptor (NMDAR) antagonist, optionally, memantine; treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126, miR-145, miR-195, miR-21, and miR-29b; and combinations thereof.

In some embodiments, the presently disclosed subject matter also relates compositions for use in treating and/or inhibiting progression of a neurological disease, condition, and/or disorder in a subject suffering from and/or at risk for developing neurological disease, condition, and/or disorder. In some embodiments, the composition comprises a reverse transcriptase inhibitor in an amount effective for treating and/or inhibiting progression of the neurological disease, condition, and/or disorder in the subject.

In some embodiments, the presently disclosed subject matter also relates compositions for use in inhibiting development of amyloid beta peptide (Aβ) in a subject suffering from Alzheimer's Disease (AD). In some embodiments the compositions comprise a reverse transcriptase inhibitor in an amount sufficient to inhibit development of Aβ in the subject.

In some embodiments, the presently disclosed subject matter also relates compositions for use in inhibiting microglial cell death, the composition comprising a reverse transcriptase inhibitor in an amount sufficient to inhibit microglial cell death. In some embodiments, the neurological disease, condition, and/or disorder is selected from the group consisting of Alzheimer's Disease (AD), cerebral amyloid angiopathy, cognitive impairment, mild cognitive impairment, Alzheimer's disease-related attention deficit symptoms, Alzheimer's disease-related neural degeneration, degenerative dementia, senile dementia, cerebral vascular dementia, alcoholic dementia, Parkinson's disease-related dementia, tic disorder, corticobasal ganglionic degeneration, and HIV-associated neurocognitive disorder (HAND), or comprises any combination thereof. In some embodiments, the reverse transcriptase inhibitor is selected from the group consisting of a nucleoside reverse transcriptase inhibitor (NRTI), a non-nucleoside reverse transcriptase inhibitor (NNRTI), and combinations thereof. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In some embodiments, the NNRTI is selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

In some embodiments, the presently disclosed compositions further comprise an additional active agent. In some embodiments, the additional active agent is selected from the group consisting of an acetylcholinesterase (AChE) inhibitor, optionally donepezil, rivastigmine, and/or galantamine; an N-methyl-d-aspartate receptor (NMDAR) antagonist, optionally, memantine; a secretase inhibitor, a beta-site APP-cleaving enzyme (BACE) inhibitor; an inhibitor of tau aggregation; an inhibitory nucleic acid, optionally an miRNA, further optionally an miRNA selected from the group consisting of miR-126, miR-145, miR-195, miR-21, and miR-29b; and combinations thereof.

In some embodiments, the presently disclosed subject matter also provides pharmaceutical compositions for treating and/or inhibiting progression of neurological diseases, conditions, and/or disorders in a subject in need thereof; and/or for inhibiting development of amyloid beta peptide (Aβ) in a subject in need thereof; and/or for inhibiting microglial cell death, optionally in a subject in need thereof and/or in vitro and/or ex vivo. In some embodiments, the pharmaceutical compositions comprise, consist essentially of, or consist of an effective amount of one or more a reverse transcriptase inhibitors.

In some embodiments, the presently disclosed subject matter also provides compositions for the preparation of medicaments for treating and/or inhibiting progression of neurological diseases, conditions, and/or disorders in a subject in need thereof; and/or for inhibiting development of amyloid beta peptide (Aβ) in a subject in need thereof; and/or for inhibiting microglial cell death, optionally in a subject in need thereof and/or in vitro and/or ex vivo. In some embodiments, the pharmaceutical compositions comprise, consist essentially of, or consist of an effective amount of one or more a reverse transcriptase inhibitors.

Accordingly, it is an object of the presently disclosed subject matter to provide methods for treating and/or inhibiting progression of a neurological disease, condition, and/or disorder in a subject suffering from and/or at risk for developing neurological disease, condition, and/or disorder. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIG. 1. Forest Plot of Incident Alzheimer's disease and NRTI exposure using a Random-Effects Model. Hazard ratios based on a Cox proportional-hazards model and adjusted for the confounding variables listed in Table 3 and in the Materials and Methods for EXAMPLE 1 presented herein below. were estimated separately for each database. The dashed vertical line denotes a hazard ratio of 1.0, which represents no difference in risk between nucleoside reverse-transcriptase inhibitor (NRTI) exposure and non-exposure. The horizontal bars represent 95% confidence intervals (CI). P values for individual databases are reported in the right-most column. An inverse-variance-weighted random-effects meta-analysis was performed to obtain a pooled estimate of the adjusted hazard ratio of incident Alzheimer's disease based on NRTI exposure. The results of the statistical test of heterogeneity using the chi-square (χ²) test statistic and its degrees of freedom (df) are shown below the plot. The Higgins I² statistic and its 95% CI are presented. The results of the statistical test of overall effect, the z test statistic, and corresponding p value are presented. *Influence of studies on meta-analysis using random-effects model. **Test for heterogeneity: χ²=6.75; df=2; p=0.0342; I²—70.4%; 95% CI=0.0% to 91.3%. Test for overall effect: z—3.79; p=0.0002.

FIG. 2. Forest Plot of Incident Alzheimer's disease and NNRTI exposure using a Random-Effects Model. Hazard ratios based on a Cox proportional-hazards model and adjusted for the confounding variables listed in Table 3 and in the Materials and Methods for EXAMPLE 1 were estimated separately for each health insurance claims database. The dashed vertical line denotes a hazard ratio of 1.0, which represents no difference in risk between non-nucleoside reverse-transcriptase inhibitor (NNRTI) exposure and non-exposure. The horizontal bars represent 95% confidence intervals (CI). P values for individual databases are reported in the right-most column. An inverse-variance-weighted random-effects meta-analysis was performed to obtain a pooled estimate of the adjusted hazard ratio of incident Alzheimer's disease based on NNRTI exposure. The results of the statistical test of heterogeneity using the chi-square (χ2) test statistic and its degrees of freedom (df) are shown below the plot. The Higgins I² statistic and its 95% CI are presented. The results of the statistical test of overall effect, the z test statistic, and corresponding P value are presented. *Influence of studies on meta-analysis using random-effects model; **Test for heterogeneity: χ²=2.59; df=2; p=0.2742; I²=22.7%; 95% CI, 0.0% to 92.0%. Test for overall effect: z=8.95, p<0.0001.

FIGS. 3A and 3B. 3TC and K-9 inhibited mouse brain microglial cell death and amyloid β-induced IL-1β release therefrom. FIG. 3A is a bar graph showing that as monitored by ELISA, amyloid β-induced IL-1β release from mouse brain microglial cell death was inhibited by 3TC and by K-9 in a dose-dependent manner. FIG. 3B is a bar graph showing that as monitoring by LDH release, mouse brain microglial cell death was inhibited by 3TC and by K-9 in a dose-dependent manner.

### DETAILED DESCRIPTION

### I. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein. In some embodiments, a subject is a human.

It is noted that all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein and for which Accession Nos. for various exemplary gene products disclosed in the GENBANK® biosequence database, are intended to encompass homologous and variant genes and gene products from humans and other animals including, but not limited to other mammals.

The methods of the presently disclosed subject matter are particularly useful for warm-blooded vertebrates. Thus, the presently disclosed subject matter concerns mammals and birds. More particularly contemplated is the isolation, manipulation, and use of stem cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of stem cells from livestock, including but not limited to domesticated swine (pigs and hogs), ruminants, horses, poultry, and the like.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

The terms “additional therapeutically active compound” or “additional therapeutic agent”, as used in the context of the presently disclosed subject matter, refer to the use or administration of a compound for an additional therapeutic use for a particular injury, disease, or disorder being treated. Such a compound, for example, could include one being used to treat an unrelated disease or disorder, or a disease or disorder which is not responsive to the primary treatment for the injury, disease or disorder being treated. Diseases and disorders being treated by the additional therapeutically active agent include, for example, hypertension and diabetes. The additional compounds can also be used to treat symptoms associated with the injury, disease, or disorder, including, but not limited to, pain and inflammation.

The term “adult” as used herein, is meant to refer to any non-embryonic or non-juvenile subject.

As used herein, an “agonist” is a composition of matter which, when administered to a mammal such as a human, enhances or extends a biological activity attributable to the level or presence of a target compound or molecule of interest in the subject.

A disease or disorder is “alleviated” if the severity of a symptom of the disease, condition, or disorder, or the frequency with which such a symptom is experienced by a subject, or both, are reduced.

As used herein, amino acids are represented by the full name thereof, by the three letter code corresponding thereto, or by the one-letter code corresponding thereto, as indicated in Table 1:

TABLE 1
Amino Acid Codes and Functionally Equivalent Codons

| | 3-Letter | 1-Letter | Functionally |
|---|---|---|---|
| Full Name | Code | Code | Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; |
| | | | CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; |
| | | | UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; |
| | | | CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue,” and can refer to a free amino acid or to an amino acid residue of a peptide. It will be apparent from the context in which the term is used whether it refers to a free amino acid or a residue of a peptide.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the subject.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)2 and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge region, thereby converting the F(ab′)₂ dimer into an Fabi monomer. The Fabi monomer is essentially a Fab with part of the hinge region. While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all intact antibody molecules.

An “antibody light chain”, as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Anti sense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, ARB, etc., to which other ingredients can be added. The term does not exclude media which have been prepared or are intended for specific uses, but which upon modification can be used for other cell types, etc.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which can be either removed from the biological system and/or chemically incorporated into the biological system.

The term “biological sample”, as used herein, refers to samples obtained from a living organism, including skin, hair, tissue, blood, plasma, cells, sweat, and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate can, for example, be redeposited as bone mineral, or by being otherwise re-utilized within the body, or excreted. “Strongly bioresorbable”, as the term is used herein, means that at least 80% of the total mass of material implanted is resorbed within one year.

The phrases “cell culture medium”, “culture medium” (plural “media” in each case), and “medium formulation” refer to a nutritive solution for cultivating cells and may be used interchangeably.

A “conditioned medium” is one prepared by culturing a first population of cells or tissue in a medium, and then harvesting the medium. The conditioned medium (along with anything secreted into the medium by the cells) can then be used in any desired way, such as to treat a disease or disorder in a subject, or to support the growth or differentiation of a second population of cells.

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cells are present in the tissue in an animal not afflicted with a disease or disorder.

A “compound”, as used herein, refers to any type of substance or agent that is commonly considered a drug, or a candidate for use as a drug, combinations, and mixtures of the above, as well as polypeptides and antibodies of the presently disclosed subject matter.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

“Chemokine”, as used herein, refers to an intercellular signaling molecule involved in the chemotaxis of white blood cells, such as T cells.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting or applying” includes administration of a compound or composition of the presently disclosed subject matter by any number of routes and approaches including, but not limited to, topical, oral, buccal, intravenous, intratumoral, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material can be shipped separately from the container with the intention that the instructional material and the compound be used cooperatively by the recipient.

Used interchangeably herein are the terms “isolate” and “select”.

The terms “isolate”, “isolated”, “isolating”, and grammatical variations thereof when used in reference to cells, refers to a single cell of interest, or a population of cells of interest, at least partially isolated from other cell types or other cellular material with which it occurs in a culture or a tissue of origin. A sample is “substantially pure” when it is in some embodiments at least 60%, in some embodiments at least 75%, in some embodiments at least 90%, and, in certain cases, in some embodiments at least 99% free of cells or other cellular material other than the cells of interest.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds.

Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule or bivalent group derived therefrom that joins two other molecules covalently or noncovalently, e.g., through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

Micro-RNAs are generally about 16-25 nucleotides in length. In some embodiments, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide can serve a non-coding function (e.g., promoter, origin of replication, ribosome-binding site, etc.), as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases, scFv are expressed on the surface of an engineered cell, for the purpose of selecting particular scFv that bind to an antigen of interest.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds”, as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest.

A “subject” of diagnosis or treatment is an animal, including a human. It also includes pets and livestock.

As used herein, a “subject in need thereof” is a patient, animal, mammal, or human, who will benefit from a method or compositions of the presently disclosed subject matter.

The term “substantially pure” describes a compound, molecule, or the like, which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, such as but not limited to in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The terminology used herein is for the purpose of describing the particular versions or embodiments only, and is not intended to limit the scope of the presently disclosed subject matter. All publications mentioned herein are incorporated by reference in their entirety.

### II. Representative Embodiments

In some embodiments, the presently disclosed subject matter relates to methods for treating and/or inhibiting progression of a neurological disease, condition, and/or disorder in a subject suffering from and/or at risk for developing neurological disease, condition, and/or disorder. As used herein, the phrase “a subject suffering from and/or at risk for developing neurological disease, condition, and/or disorder” is used interchangeably with the phrase “a subject in need thereof”. Exemplary, non-limiting neurological diseases, conditions, and/or disorders that can be treated with the methods and compositions of the presently disclosed subject matter include, but are not limited to Alzheimer's Disease (AD), cerebral amyloid angiopathy, cognitive impairment, mild cognitive impairment, Alzheimer's disease-related attention deficit symptoms, Alzheimer's disease-related neural degeneration, degenerative dementia, senile dementia, cerebral vascular dementia, alcoholic dementia, Parkinson's disease-related dementia, tic disorder, corticobasal ganglionic degeneration, and HIV-associated neurocognitive disorder (HAND), as well as any combinations thereof.

In some embodiments, the presently disclosed subject matter relates to methods for of inhibiting development amyloid beta peptide (Aβ) in a subject suffering from Alzheimer's Disease (AD.

In some embodiments, the presently disclosed subject matter relates to methods for inhibiting microglial cell death.

In some embodiments, the presently disclosed methods comprise administering to the subject suffering from and/or at risk for developing neurological disease, condition, and/or disorder; or who has an accumulation of amyloid beta peptide (Aβ) and/or is at risk for developing an accumulation of amyloid beta peptide (Aβ), and/or who has developed and/or is at risk for experiencing microglial cell death a composition comprising a reverse transcriptase inhibitor in an amount and via a route effective for treating and/or inhibiting progression of the neurological disease, condition, and/or disorder in the subject. Any reverse transcriptase inhibitor can be employed in the methods of the presently disclosed subject matter. In some embodiments, the reverse transcriptase inhibitor selected from the group consisting of a nucleoside reverse transcriptase inhibitor (NRTI), a non-nucleoside reverse transcriptase inhibitor (NNRTI), or any combination thereof.

In some embodiments, the reverse transcriptase inhibitor (RTI) is an NRTI. Exemplary NRTIs include abacavir ((1S,4R)-4-[2-amino-6-(cyclopropylamino)-9H-purin-9-yl]cyclopent-2-en-1-yl}methanol; ABC; U.S. Pat. No. 8,183,370), adefovir ({[2-(6-amino-9H-purin-9-yl)ethoxy]methyl}phosphonic acid; bis-POM PMEA; U.S. Pat. No. 5,663,159), amdoxovir ([(2R,4R)-4-(2,6-diaminopurin-9-yl)-1,3-dioxolan-2-yl]methanol; Murphy et al. (2010) Antivir Ther 15(2):185-192), apricitabine (4-amino-1-[(2R,4R)-2-(hydroxymethyl)-1,3-oxathiolan-4-yl]pyrimidin-2(1H)-one; AVX754; PCT International Patent Application Publication No. WO 2014/183147), censavudine (1-[(2R,5R)-5-ethynyl-5-(hydroxymethyl)-2H-furan-2-yl]-5-methylpyrimidine-2,4-dione; U.S. Pat. Nos. 7,589,078; 8,193,165; 9,126,971), didanosine (9-((2R,5S)-5-(hydroxymethyl)tetrahydrofuran-2-yl)-3H-purin-6(9H)-one; DDI; U.S. Pat. Nos. 7,589,078; 8,193,165; 9,126,971), elvucitabine (4-amino-5-fluoro-1-[(2S,5R)-5-(hydroxymethyl)-2,5-dihydrofuran-2-yl]pyrimidin-2-one; U.S. Patent Application Publication No. 2011/0150997), emtricitabine (2′,3′-dideoxy-5-fluoro-3′-thiacytidine 4-amino-5-fluoro-1-[(2R,5 S)-2-(hydroxymethyl)-1,3-oxathiolan-5-yl]-1,2-dihydropyrimidin-2-one; FTC; PCT International Patent Application Publication No. WO 2014/176532), entecavir (2-Amino-9-[(1S,3R,4S)-4-hydroxy-3-(hydroxymethyl)-2-methyl-idenecyclopentyl]-1H-purin-6-one; ETV; U.S. Pat. No. 6,627,224), lamivudine (2′,3′-di deoxy-3′-thiacytidine-4-Amino-1-[(2R,5 S)-2-(hydroxymethyl)-1,3-oxathiolan-5-yl]-1,2-dihydropyrimidin-2-one; 3TC; U.S. Pat. No. 8,481,554), racivir (4-amino-5-fluoro-1-[(2 S,5R)-2-(hydroxymethyl)-1,3-oxathiolan-5-yl]-1,2-dihydropyrimidin-2-one; Otto (2004) Curr Opin Pharmacol 4(5):431-436), stampidine (methyl N-((4-bromophenoxy){[(2S,5R)-5-(5-methyl-2,4-dioxo-3,4-dihydropyrimidin-1(2H)-yl)-2,5-dihydrofuran-2-yl]methoxy}phosphoryl)-D-alaninate; U.S. Pat. No. 6,350,736), stavudine (1-[(2R,5 S)-5-(hydroxymethyl)-2,5-dihydrofuran-2-yl]-5-methyl-1,2,3,4-tetrahydropyrimidine-2,4-dion; d4T; U.S. Pat. No. 8,026,356), tenofovir disoproxil (Bis{[(isopropoxycarbonyl)oxy]methyl}({[(2R)-1-(6-amino-9H-purin-9-yl)-2-propanyl]oxy}methyl)phosphonate; TDF; PCT International Patent Application Publication No. WO 2008/007382), tenofovir alafenamide (Isopropyl (2S)-2-[[[(1R)-2-(6-aminopurin-9-yl)-1-methyl-ethoxy]methyl-phenoxy-phosphoryl]amino]propanoate; GS-7340; U.S. Pat. No. 9,296,769), zalcitabine (4-amino-1-((2R,5S)-5-(hydroxymethyl)tetrahydrofuran-2-yl)pyrimidin-2(1H)-one; ddC; Shelton et al. (1993) Ann Pharmacother 27(4):480-489), zidovudine (ZDV)/azidothymidine (3′-deoxy-3′-azidothymidine 1-[(2R,4S,5 S)-4-Azido-5-(hydroxymethyl)oxolan-2-yl]-5-methylpyrimidine-2,4-dione; AZT; U.S. Pat. Nos. 5,905,082; 6,294,540; 6,417,191), derivatives thereof, optionally alkylated derivatives thereof, further optionally tri-methoxy-3TC (also known as Kamuvudine-9 and K-9), pharmaceutically acceptable salts thereof, and combinations thereof. See also U.S. Patent Application Publication Nos. 2019/0022115, 2019/0055273, 2019/0177326, 2019/0185508. Each of these U.S. patents and patent applications publications is incorporated by reference in its entirety.

In some embodiments, the reverse transcriptase inhibitor (RTI) is an NNRTI. Exemplary NNRTIs include delavirdine (N-[2-({4-[3-(propan-2-ylamino)pyridin-2-yl]piperazin-1-yl}carbonyl)-1H-indol-5-yl]methanesulfonamide; DLV; U.S. Pat. No. 5,563,142), efavirenz ((S)-6-chloro-4-(Cyclopropylethynyl)-1,4-dihydro-(S)-6-chloro-4-(cyclopropylethynyl)-1,4-dihydro-4-(trifluoromethyl)-2H-3,1-benzoxazin-2-one-4-(trifluoromethyl)-2H-3,1-benzoxazin-2-one; EFV; U.S. Pat. Nos. 5,811,423; 5,814,639; 5,914,331; 6,043,230; 6,238,695; 6,555,133; 6,639,071; 6,939,964), etravirine (4-({6-amino-5-bromo-2-[(4-cyanophenyl)amino]pyrimidin-4-yl}oxy)-3,5-dimethylbenzonitrile; ETR; U.S. Pat. Nos. 6,878,717; 7,037,917; 7,887,845; 8,003,789), nevirapine (2-cyclopropyl-7-methyl-2,4,9,15-tetraazatricyclo[9.4.0.0^{3,8}]pentadeca-1(11),3,5,7,12,14-hexaen-10-on; NVP; U.S. Pat. Nos. 5,366,972; 8,460,704), rilpivirine (4-{[4-({4-[(1E)-2-cyanoeth-1-en-1-yl]-2,6-dimethylphenyl}amino)pyrimidin-2-yl]amino}benzonitrile; TMC278; 5,814,639; 5,914,331; 5,922,695; 5,935,946; 5,977,089; 6,043,230; 6,642,245; 6,703,396; 8,592,397; 9,242,986), doravirine (3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile; MK-1439; U.S. Pat. No. 8,486,975), derivatives thereof, pharmaceutically acceptable salts thereof, and combinations thereof. See also U.S. Patent Application Publication No. 2016/0287568. Each of these U.S. patents and patent applications publications is incorporated by reference in its entirety.

In some embodiments, the methods of the presently disclosed subject matter are employed in vivo, ex vivo, and/or in vitro. By way of example and not limitation, a method for treating and/or inhibiting progression of a neurological disease, condition, and/or disorder in a subject suffering from and/or at risk for developing neurological disease, condition, and/or disorder, for inhibiting development amyloid beta peptide (Aβ) in a subject suffering from Alzheimer's Disease (AD), and/or for inhibiting microglial cell death can be employed in vivo, ex vivo, and/or in vitro.

In some embodiments of the presently disclosed methods, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

In some embodiments, suitable formulations can also include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The methods of the presently disclosed subject matter can be used with additional adjuvants or biological response modifiers including, but not limited to, cytokines and other immunomodulating compounds.

In some embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies when using the compositions described herein. Drugs useful in the presently disclosed subject matter may, for example, possess a pharmaceutical property selected from the group consisting of antimitotic, antikinase, alkylating, antimetabolite, antibiotic, alkaloid, anti-angiogenic, pro-apoptotic agents, and combinations thereof.

The compositions of the presently disclosed subject matter can be administered by any route of administration reasonably expected to deliver the compositions to a desired target site. Suitable methods for administration of the compositions of the presently disclosed subject matter thus include, but are not limited to intravenous administration and delivery directly to the target tissue or organ. In some embodiments, the method of administration encompasses features for regionalized delivery or accumulation of the to composition at the site in need of treatment. In some embodiments, the composition is/are delivered directly into the nervous system. In some embodiments, selective delivery of the composition is accomplished by intravenous injection of composition, where they accumulate in the nervous system (e.g., the brain). Other modes of administration that can be employed include topical, oral, buccal, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means. Compounds or agents of the presently disclosed subject matter can be administered to a subject by one or more of these routes when appropriate.

Where the administration of the composition is by injection or direct application, the injection or direct application may be in a single dose or in multiple doses. Where the administration of the composition is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient(s), the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, ed., 1985, Remington's Pharmaceutical Sciences, Mack Publishing Co., Easton, Pa., United States of America, which is incorporated herein by reference.

An effective dose of a composition of the presently disclosed subject matter is administered to a subject in need thereof. A “treatment effective amount”, “therapeutic amount”, or “therapeutically effect amount” is an amount of a therapeutic composition sufficient to produce a measurable response (e.g., a biologically or clinically relevant response in a subject being treated). In some embodiments, an activity that inhibits a viral infection is measured. Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using generally applicable assay methods, one skilled in the art can readily assess the potency and efficacy of a candidate compound of the presently disclosed subject matter and adjust the therapeutic regimen accordingly. After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

As such, in some embodiments the presently disclosed composition thereof is/are present in a pharmaceutically acceptable carrier, which in some embodiments can be a pharmaceutically acceptable for use in humans.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In some embodiments, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the active agent(s) encapsulated in nanoparticles and/or microparticles (including but not limited to liposomes). The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

Compositions and methods for encapsulating active agents in nanoparticles and/or microparticles (including but not limited to liposomes) are disclosed, for example, in U.S. Pat. No. 9,867,888 and U.S. Patent Application Publication Nos. 2018/0140717, 2018/0147298, 2018/0148719, 2018/0177727, 2018/0221402, and 2019/0345492, each of which is incorporated herein by reference in its entirety.

In some embodiments, the presently disclosed methods further comprise administering to the subject an additional treatment, optionally an additional AD treatment. In some embodiments, the additional AD treatment is selected from the group consisting of treatment with an acetylcholinesterase (AChE) inhibitor, optionally donepezil (2-[(1-benzylpiperidin-4-yl)methyl]-5,6-dimethoxy-2,3-dihydro-1H-inden-1-one; U.S. Pat. Nos. 4,895,841; 7,727,548; 7,727,552; 8,039,009; 8,173,708; 8,283,379; 8,329,752; 8,362,085), rivastigmine (3-[(1 S)-1-(dimethylamino)ethyl]phenyl N-ethyl-N-methylcarbamate; U.S. Pat. Nos. 4,948,807; 6,316,023; 6,335,031), and/or galantamine ((1 S,12 S,14R)-9-methoxy-4-methyl-11-oxa-4-azatetracyclo[8.6.1.0^{1,12}.0^{6,17}]heptadeca-6(17),7,9,15-tetraen-14-ol; U.S. Pat. Nos. 6,099,863; 6,358,527; 7,160,559); treatment with an N-methyl-d-aspartate receptor (NMDAR) antagonist, optionally, memantine (3,5-dimethyladamantan-1-amine; U.S. Pat. Nos. 8,039,009; 8,058,291; 8,168,209; 8,173,708; 8,283,379; 8,329,752; 8,338,486; 8,362,085; 8,598,233); treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126, miR-145, miR-195, miR-21, and miR-29b (see e.g., Wang et al. (2019) Front Genet. 10:153); and combinations thereof. See also U.S. Pat. No. 9,365,647 and U.S. Patent Application Publication Nos. 2018/0177790 and 2020/0071699, which along with the references cited hereinabove are incorporated by reference in their entireties.

In some embodiments, the presently disclosed subject matter provides use of pharmaceutical compositions comprising, consisting essentially of, or consisting of an effective amount of a composition comprising one or more a reverse transcriptase inhibitors to treat and/or inhibit progression of neurological diseases, conditions, and/or disorders in a subject in need thereof; and/or to inhibit development of amyloid beta peptide (Aβ) in a subject in need thereof; and/or to inhibit microglial cell death, optionally in a subject in need thereof and/or in vitro and/or ex vivo.

In some embodiments, the presently disclosed subject matter provides use of an effective amount of a composition comprising one or more a reverse transcriptase inhibitors for the preparation of a medicament to treat and/or inhibit progression of neurological diseases, conditions, and/or disorders in a subject in need thereof; and/or to inhibit development of amyloid beta peptide (Aβ) in a subject in need thereof; and/or to inhibit microglial cell death, optionally in a subject in need thereof and/or in vitro and/or ex vivo.

The presently disclosed subject matter also relates in some embodiments to compositions for use in the presently disclosed methods. In some embodiments, the compositions comprise a reverse transcriptase inhibitor in an amount effective for treating and/or inhibiting progression of the neurological disease, condition, and/or disorder in the subject, for inhibiting development of amyloid beta peptide (Aβ) in a subject suffering from Alzheimer's Disease (AD), and/or for inhibiting microglial cell death, the composition comprising a reverse transcriptase inhibitor in an amount sufficient to inhibit microglial cell death. In some embodiments, the compositions of the presently disclosed subject matter are employed to treat and/or inhibit progression of Alzheimer's Disease (AD), cerebral amyloid angiopathy, cognitive impairment, mild cognitive impairment, Alzheimer's disease-related attention deficit symptoms, Alzheimer's disease-related neural degeneration, degenerative dementia, senile dementia, cerebral vascular dementia, alcoholic dementia, Parkinson's disease-related dementia, tic disorder, corticobasal ganglionic degeneration, and/or HIV-associated neurocognitive disorder (HAND), or any combinations thereof.

In some embodiments, the presently disclosed compositions and uses comprise a reverse transcriptase inhibitor is selected from the group consisting of a nucleoside reverse transcriptase inhibitor (NRTI), a non-nucleoside reverse transcriptase inhibitor (NNRTI), and combinations thereof. The NRTIs and NNRTIs disclosed herein, as well as any other NRTIs and/or NNRTIs whether known or not yet developed can be employed in the compositions of the presently disclosed subject matter.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for Example 1

Databases. Veterans Health Administration (VA): claims from the United States VA system from January 2000-July 2017 were employed. Data were extracted from the VA Informatics and Computing Infrastructure (VINCI). Data include all inpatient, outpatient, and pharmacy claims. The completeness, utility, accuracy, validity, and access methods are described on the VA website. PearlDiver: Data on patients with commercial health insurance was obtained from the PearlDiver Patient Records Database (Colorado Springs, Colorade, United States of America), which captures health care claims, medication usage, and laboratory data for persons in the Humana network from the first quarter of 2007 to the third quarter of 2017. All data within the PearlDiver database are compliant with the United States Health Insurance Portability and Accountability Act (HIPPA) and were thus deemed exempt from institutional review board's approval by the University of Virginia Institutional Review Board. Truven: Data was obtained from the Truven Health Analytics MARKETSCAN® brand Commercial Database, which captures employer-based health care claims and medication usage for the period from January 2006-December 2017. All data are HIPPA-compliant and were thus deemed exempt from institutional review board's approval by the University of Virginia Institutional Review Board.

Participants and Sample Selection. Patients were included in the analysis if they met these criteria: had at least 2 diagnoses of HIV/AIDS or hepatitis B during the study. Individuals with pre-existing Alzheimer's disease (≥1 diagnosis prior to diagnosis of HIV or hepatitis B) were excluded.

Exposure to NRTIs. Individuals were classified as receiving NRTI medications if they filled ≥1 outpatient pharmacy prescription for these medications as identified based on American Hospital Formulary Service drug codes and U.S. National Drug Codes.

Dependent Variable. Time to initial diagnosis of Alzheimer's disease during the follow-up period, as identified by the ICD-9-CM code (331.0) and ICD-10-CM codes (G30.x), was the dependent variable for this analysis. Patients with Alzheimer's disease prior to index HIV or hepatitis B diagnosis were excluded. Patients were followed until the first date of 1) Alzheimer's disease, 2) death, 3) end date of dataset.

Analysis. Unadjusted odds ratios and 95% confidence intervals were estimated with the use of bivariate logistic regression with the occurrence of Alzheimer's disease as the dependent variable and exposure to NRTIs as the key explanatory variable. The proportions of subjects with Alzheimer's disease in each group were compared with the use of chi-square tests. To analyze the risk of Alzheimer's disease between those exposed to NRTIs and those not exposed to NRTI medications, Cox proportional hazard models. The adjusted model included were fit adjusted as covariates demographic variables (age, race, sex), comorbidities (Charlson comorbidity index, tobacco use, and the 18 categories of diseases listed in Table 3), and laboratory test values (CD4 counts, viral load, body mass index) known to be associated with Alzheimer's disease including those listed by the Alzheimer's Association, Alzheimer Society of Canada, BrightFocus Foundation, U.S. National Institute on Aging, World Alzheimer Report 2018, and those identified by supplementary literature research on PubMed; 95% confidence intervals for hazard ratios were constructed based on standard errors derived from the model. Race and laboratory tests were available in the PearlDiver and Veterans datasets. Statistical tests were two-sided. p values <0.05 were considered statistically significant.

Meta-analysis. An inverse, variance-weight meta-analysis of the three databases was performed to estimate the combined unadjusted odds ratio (OR) and adjusted hazard ratio (HR) and to compute 95% confidence intervals (CI) using a random-effects model. Analyses were performed with the use of the statistical program R, version 3.5.1 (the Comprehensive R Archive Network (CRAN) project and the R package meta. The restricted maximum-likelihood estimator method to estimate the between-study variance. A forest plot was created to depict the HR and 95% CI of each study and of the pooled results. Variability among databases was indicated by tests for homogeneity. Hence, results of the random-effects model are presented. The assumption of heterogeneity is reasonable because of differential clinical settings, clinical and sociodemographic characteristics, and time-periods across the four databases. A random-effects model is most appropriate as it assumes that individual databases are samples of different populations with different underlying true effects, unlike fixed-effects models, which assume that individual databases are samples from the same population.

### Example 1

### Multi-Centered Study of NRTI Use and Alzheimer's Disease

A multi-centered study of three of the largest health insurance claims databases in the United States to analyze the relationship between use of nucleoside reverse transcriptase inhibitors (NRTIs), drugs that are used to treat HIV-1 and hepatitis B infections, and development of Alzheimer's disease among HIV- or hepatitis B-infected individuals 55 years of age or older with no prior diagnosis of Alzheimer's disease. Disease-specific diagnoses using codes from the International Classification of Diseases, Clinical Modification, Ninth and Tenth Revisions and outpatient pharmacy prescriptions using American Hospital Formulary Service drug codes and U.S. National Drug Codes were employed. Unadjusted odds ratios were estimated using bivariate logistic regression with the occurrence of Alzheimer's disease as the dependent variable and exposure to NRTIs as the key explanatory variable. In each of the three databases, there was a reduced incident Alzheimer's disease among NRTI users (Table 2).

TABLE 2
NRTI Use and Incident Alzheimer's Disease

| | NRTI untreated | NRTI treated | Odds Ratio | |
|---|---|---|---|---|
| Database | (Cases/Total) | (Cases/Total) | (95% CI) | p value |
| PearlDiver Humana | 182/5,536 | 51/4,375 | 0.34 | <0.0001 |
| (2007-17) | | | (0.25, 0.46) | |
| Veterans Health | 153/21,046 | 118/42,248 | 0.38 | <0.0001 |
| Administration | | | (0.30, 0.49) | |
| (2000-17) | | | | |
| Truven MarketScan | 6/2,039 | 10/15,138 | 0.22 | 0.0038 |
| Commercial Claims | | | (0.08, 0.62) | |
| (2006-17) | | | | |
| Pooled | 341/28,441 | 179/61,761 | 0.36 | <0.0001 |
| | | | (0.30, 0.43) | |

Overall, in a pooled analysis, incident development of Alzheimer's disease was significantly lower among individuals exposed to NRTIs than among individuals not prescribed NRTIs (unadjusted odds ratio, 0.36; 95% CI, 0.30 to 0.43; p<0.0001).

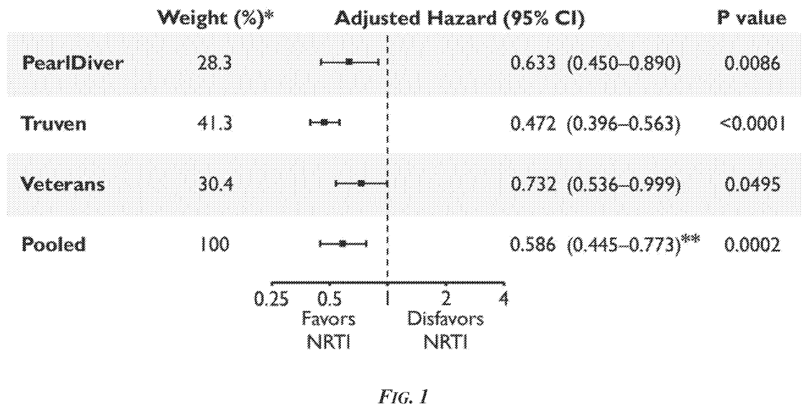

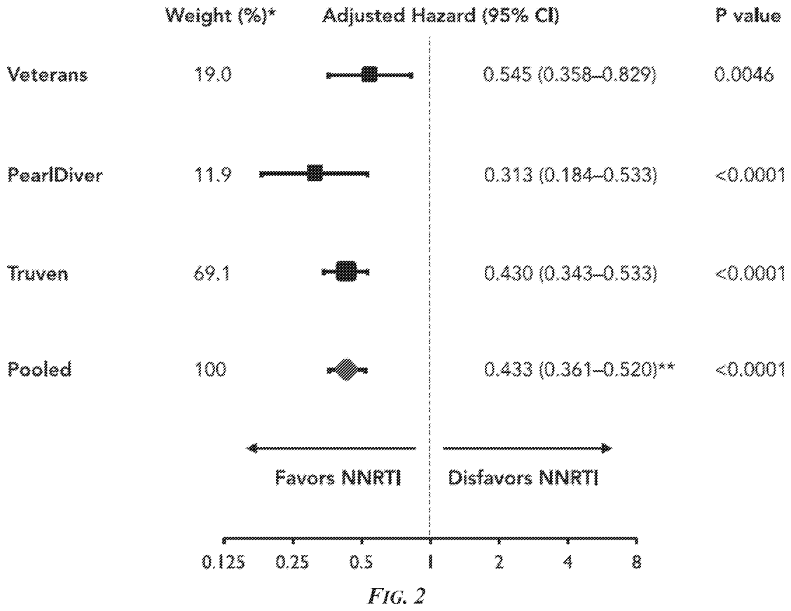

Cox proportional hazards regression models, adjusting for sociodemographic variables (age, gender, race, smoking), comorbidities reported to be associated with Alzheimer's disease (Table 3), overall health (Charlson comorbidity index), viral load, and CD4⁺ cell count, showed a reduced risk of Alzheimer's disease among NRTI users (FIG. 1) and among NNRTI users (FIG. 2) in each of the three databases. An inverse, variance-weight meta-analysis of these adjusted analyses using a random-effects model showed that collectively, among 90,202 patients aged 55 years and above and diagnosed with HIV-1 or hepatitis B, the adjusted risk of incident Alzheimer's disease was 41% lower in patients with NRTI exposure (adjusted hazard ratio, 0.586; 95% CI, 0.445 to 0.773; p=0.0002). A similar meta-analysis revealed that the adjusted risk of incident Alzheimer's disease was 57% lower in patients with NNRTI exposure (adjusted hazard ratio, 0.433; 95% CI, 0.361 to 0.520; p<0.0001).

TABLE 3
Comorbidities Reported to be Associated with Alzheimer's Disease
Comorbidities Associated with Alzheimer's Disease Development

| | | |
|---|---|---|
| Alcohol dependence | Generalized anxiety | Ischemic Heart disease |
| syndrome | disorder | |
| Atrial fibrillation | Hyperlipidemia | Other Heart Disease |
| Cerebral infarction | Hypertensive diseases | Parkinson's disease |
| Chronic Kidney disease | Hyperthyroidism | Pure hypercholesterolemia |
| Depression | Hypertriglyceridemia | Traumatic brain injury |
| Down syndrome | Hypothyroidism | Type 2 diabetes |

HIV-affected patients often, but not always, are exposed to both NRTI and NNRTI medications as well as protease inhibitors (PIs). Therefore, additional Cox proportional hazards regression analyses were performed wherein exposure to NRTIs, NNRTIs, and PIs were included in the model, and, as above, adjusted for all the other sociodemographic variables, comorbidities, overall health status, and laboratory values. In this combined drug class analysis, it was found that NRTI use was associated with reduced incident development of Alzheimer's disease in one database but not in the other two databases (Table 4). When pooled, the association between NRTI use and reduced incident development of Alzheimer's disease was found when a fixed-effect model was used for meta-analysis but not when a random-effects model was used.

TABLE 4
NRTI Use Association with Reduced Incident
Development of Alzheimer's Disease

| | Weight (%) | Weight (%) | Adjusted | |
|---|---|---|---|---|
| | (Fixed- | (Random- | Hazard | |
| Database | Effects)* | Effects)** | (95% CI) | p value |
| Veterans | 31.7 | 33.9 | 0.858 | 0.293 |
| | | | (0.649-1.135) | |
| Truven | 51.3 | 35.9 | 0.550 | <0.0001 |
| | | | (0.441-0.685) | |
| PearlDiver | 17.0 | 30.2 | 1.183 | 0.389 |
| | | | (0.807-1.735) | |
| Pooled | 100 | | 0.721 | <0.0001 |
| (Fixed- | | | (0.616-0.844) | |
| Effects) | | | | |
| Pooled | | 100 | 0.806 | 0.331 |
| (Random- | | | (0.522-1.245) | |
| Effects) | | | | |

*Influence of studies on meta-analysis using fixed-effects model
**Influence of studies on meta-analysis using random-effects model
**Test for heterogeneity: χ² = 13.73, df = 2, p = 0.001, I² = 85.4%; 95% CI, 57.3% to 95.0%.
Test for overall effect using fixed-effects model: z = 4.06, p <0.0001
Test for overall effect using random-effects model: z = 0.97, p = 0.331

In contrast, the association between NNRTI use and reduced incident development of Alzheimer's disease persisted in all three databases, individually and when pooled, both in a fixed-effect model and a random-effect model (Table 5). Thus, the association for lower incident Alzheimer's disease is more robust for NNRTIs than NRTIs.

TABLE 5
NNRTI Use Association with Reduced Incident
Development of Alzheimer's Disease

| | Weight (%) | Weight (%) | Adjusted | |
|---|---|---|---|---|
| | (Fixed- | (Random- | Hazard | |
| Database | Effects)* | Effects)** | (95% CI) | p value |
| Veterans | 20.3 | 20.3 | 0.620 | 0.0388 |
| | | | (0.393-0.976) | |
| Truven | 67.0 | 67.0 | 0.580 | <0.0001 |
| | | | (0.451-0.746) | |
| PearlDiver | 12.7 | 12.7 | 0.366 | 0.0006 |
| | | | (0.206-0.650) | |
| Pooled | 100 | | 0.555 | <0.0001 |
| (Fixed- | | | (0.452-0.680) | |
| Effects) | | | | |
| Pooled | | 100 | 0.555 | <0.0001 |
| (Random- | | | (0.452-0.680) | |
| Effects) | | | | |

*Influence of studies on meta-analysis using fixed-effects model
**Influence of studies on meta-analysis using random-effects model
**Test for heterogeneity: χ² = 2.37, df = 2, p = 0.306, I² = 15.7%; 95% CI, 0.0% to 91.2%.
Test for overall effect using fixed-effects model: z = 5.65, p <0.0001
Test for overall effect using random-effects model: z = 5.65, p <0.0001

### Example 2

### 3TC and by K-9 Inhibited Mouse Brain Microglial Cell Death and Amyloid B-Induced IL-1β Release Therefrom

Primary microglial cultures were prepared as follows. Briefly, newborn wild type (WT) mice (P1-P4) were perfused with ice-cold 1×PBS with heparin (Sigma-Aldrich, St. Louis, Mo., United States of America). Meninges were removed from the brains followed by mechanical dissociation using a Dounce homogenizer. Brain homogenate was subjected to enzymatic dissociation by 0.25% trypsin (Thermo Fisher Scientific, Inc., Waltham, Mass., United States of America) and cells were filtered using a 70 μM cell strainer. Then, cells were resuspended in DMEM (Thermo Fisher Scientific) supplemented with 10% FBS and 100 U/mL penicillin-streptomycin (Thermo Fisher Scientific) and were seeded into pre-coated T75 tissue culture flasks. After 4 days, the media was replaced with fresh microglia media containing 25% L-cells and cell culture medium was renewed every 3 days. Microglial cultures were used after 14 days of primary cultivation. They were collected by gently shaking (250 rpm for 1 hour at 37° C.) the flasks. For cell culture experiments, microglia were allowed to re-attach to the cell culture dish overnight before use.

Aβ 1-42 (1 mg, AnaSpec Inc., San Jose, Calif., United States of America) lyophilized powder was dissolved in 222 μL in 100% hexafluoroisopropanol and aliquoted into 5 Eppendorf tubes with lids open overnight in a cell culture hood. On the next day, excess moisture was removed by drying the tubes in a speed vac at 4° C. for 1 hour. 5 mM Aβ 1-42 stocks were prepared by adding 40 μL of DMSO (Sigma-Aldrich) followed by vortex and sonication for 10 minutes. To oligomerize Aβ 1-42 peptides were resuspended in DMEM F/12 (Sigma-Aldrich) without phenol red followed by incubation on a rotary machine at 4° C. for 48 hours.

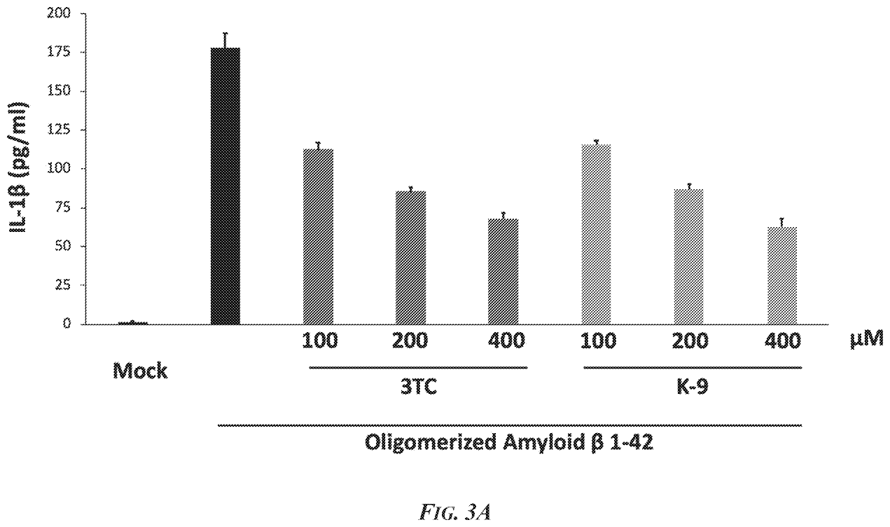

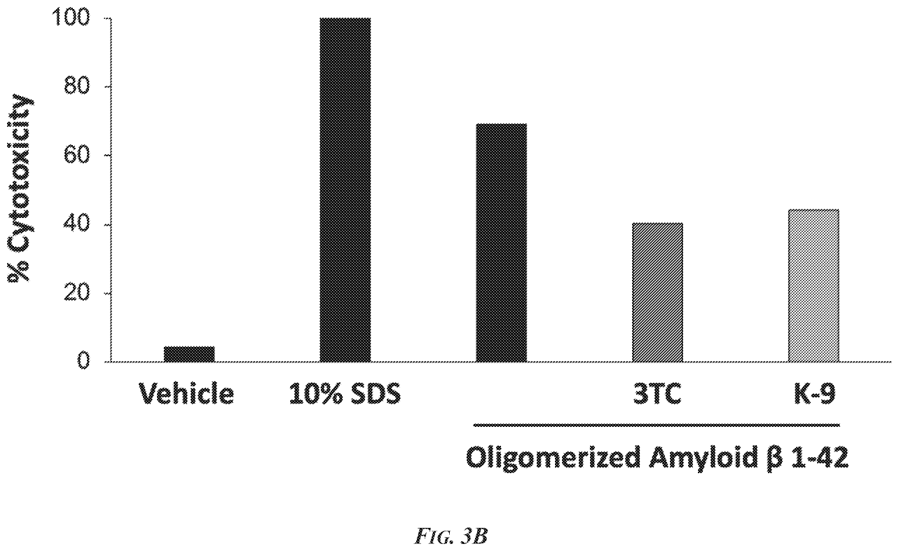

Microglia were pre-treated with NRTI's Lamivudine (3TC) or tri-methoxy 3TC (K9) at desired concentrations for 1 hour. Cells were primed with Pam3CSK (1 μg/μL) for 12 hours, followed by stimulation with 10 μM Aβ 1-42 for 6 hours. In addition, Pam3CSK-primed microglia were transfected with 5 μg/mL LPS using Lipofectamine 2000 (Invitrogen Corp. Carlsbad, Calif., United States of America) according to manufacturer's instructions. 0.8 million cells/mL were seeded per well in 6-well cell culture dishes overnight. Secreted mouse IL-10 in the medium were detected by ELISA (Mouse IL-1β DuoSet, R&D Systems, Minneapolis, Minn., United States of America, Catalog No. DY401) according to the manufacturer's instructions. Serum free media (DMEM+100 U/mL penicillin-streptomycin) was used throughout the ELISA. The results are presented in FIG. 3A.

Pam3CSK primed microglia (10,000 cells/well) were seeded in a 96-well plate with or without of Aβ 1-42 or LPS in the presence or absence of 3TC or K9 (100 After incubation for 24 hours, supernatant was collected for the detection of LDH release by using an LDH cytotoxicity detection kit (CYTOTOX 96® brand nonradioactive cytotoxicity assay; Promega Corp., Madison, Wis., United States of America) according to the manufacturer's instruction. Then the OD value was measured at a 490 nm wavelength using a plate reader. The results are presented in FIG. 3B.

### Discussion of the Examples

Some studies have reported an increased prevalence of amyloid-β plaques in the postmortem brain sections of HIV-infected patients (Esiri et al., 1998; Green et al., 2005; Rempel & Pulliam, 2005), although patients, any relationship to anti-HIV treatment regimens was not studied.

There are two classes of reverse transcriptase inhibitors: NRTIs and NNRTIs. NRTIs are competitive, promiscuous inhibitors of many reverse transcriptases including both viral reverse transcriptases and human reverse transcriptases (LINE-1, telomerase). In contrast, NNRTIs are non-competitive, allosteric inhibitors of HIV-1 reverse transcriptase. NNRTIs do not inhibit human telomerase, and they do not inhibit human LINE-1, except for a few NNRTIs and that too at very high concentrations that are not achieved by current clinical dosing. NNRTIs, as well as NRTIs, do block human endogenous retroviruses (HERVs).

Activation of the inflammasome has been implicated in Alzheimer's disease; NRTIs block inflammasome but NNRTIs do not block inflammasome. The results described herein are unexpected, and that NNRTIs also could be useful in this disease because of the many differences in their mode of action. Furthermore, in the most rigorous analysis conducted, it was unexpectedly found that NNRTIs are associated with the most protection against Alzheimer's disease in the health insurance claims databases, whereas NRTIs were not.

These data do provide additional support for exploring mechanisms whereby NRTIs might be beneficial in this context. In addition to a direct effect on reverse transcriptase activity generating APP gencDNA, NRTIs also could inhibit activation of the NLRP3 inflammasome (Fowler et al., 2104), which is implicated in Alzheimer's disease (Heneka et al., 2013). This dual mode of action could be salient in antagonizing Alu, HERV, and L1 retrotransposon transcripts, which are enriched in Alzheimer's disease brains (Guo et al., 2018), as these RNAs utilize reverse transcriptase as a key component of their lifecycle (Kazazian, Jr. & Moran, 2017), and some of them also activate the inflammasome (Tarallo et al., 2012; Kerur et al., 2018). NRTIs also could inhibit enhanced L1 reverse transcriptase activity in senescent cells (De Cecco et al., 2019), some of which are more abundant in Alzheimer's disease brains (Bhat et al., 2012).

In summary, presented herein is evidence that NRTI and NNRTI use is associated with a reduced incident Alzheimer's disease among older HIV- or hepatitis B-infected individuals. It is unknown whether this protective association would exist among individuals not infected by these viruses. In addition, given the severe toxicity of first-generation NRTIs and the occasional serious adverse effects reported with current-generation NRTIs, it would be interesting to test alkylated NRTIs known as Kamuvudines (Fowler et al., 2104; De Cecco et al., 2019; see also U.S. Patent Application Publication No. 2019/0262341), which block inflammasome activation without inducing the mitochondrial toxicity associated with NRTIs, in a prospective clinical trial.

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
Bhat et al. (2012) Astrocyte senescence as a component of Alzheimer's disease. PLoS One 7:e45069.
Bird et al. (1988) Single-chain antigen-binding proteins. Science 242:423-426.
De Cecco et al. (2019) L1 drives IFN in senescent cells and promotes age-associated inflammation. Nature 566:73-78.
Esiri et al. (1998) Prevalence of Alzheimer plaques in AIDS. J Neurol Neurosurg Psychiatry 65:29-33.
Fowler et al. (2104) Nucleoside reverse transcriptase inhibitors possess intrinsic anti-inflammatory activity. Science 346:1000-1003.
Genaro, ed., 1985, Remington's Pharmaceutical Sciences, Mack Publishing Co., Easton, Pa., United States of America.
Green et al. (2005) Brain deposition of beta-amyloid is a common pathologic feature in HIV positive patients. AIDS 19:407-411.
Gross & Mienhofer (1981) The Peptides, Vol. 3, Academic Press, New York, N.Y., United States of America, pp. 3-88.
Guo et al. (2018) Tau Activates Transposable Elements in Alzheimer's Disease. Cell Rep 23:2874-2880.
Heneka et al. (2013) NLRP3 is activated in Alzheimer's disease and contributes to pathology in APP/PS1 mice. Nature 493:674-678.
Huston et al. (1988) Protein engineering of antibody binding sites: recovery of specific activity in an anti-digoxin single-chain Fᵥ analogue produced in Escherichia coli. Proc Natl Acad Sci USA 85:5879-5883.
Kazazian, Jr. & Moran (2017) Mobile DNA in Health and Disease. N Engl J Med 377:361-370.
Kerur et al. (2018) cGAS drives noncanonical-inflammasome activation in age-related macular degeneration. Nat Med 24:50-61.
Lee et al. (2018) Somatic APP gene recombination in Alzheimer's disease and normal neurons. Nature 563:639-645.
Otto (2004) New nucleoside reverse transcriptase inhibitors for the treatment of HIV infections. Curr Opin Pharmacol 4(5):431-436
PCT International Patent Application Publication Nos. WO 2008/007382, WO 2014/176532, WO 2014/183147.
Rempel & Pulliam (2005) HIV-1 Tat inhibits neprilysin and elevates amyloid beta. AIDS 19:127-135.
Shelton et al. (1993) Zalcitabine. Ann Pharmacother 27(4):480-489.
Tarallo et al. (2012) DICER1 loss and Alu RNA induce age-related macular degeneration via the NLRP3 inflammasome and MyD88. Cell 149:847-859.
Turner et al. (2016) An individual with human immunodeficiency virus, dementia, and central nervous system amyloid deposition. Alzheimers Dement (Amst) 4:1-5.
U.S. Patent Application Publication Nos. 2003/0017534; 2011/0150997; 2016/0287568; 2018/0140717; 2018/0147298; 2018/0148719; 2018/0177727; 2018/0177790; 2018/0221402; 2018/0298087; 2018/0312588; 2018/0346564; 2019/0022115; 2019/0055273; 2019/0151448; 2019/0177326; 2019/0185508; 2019/0262341; 2019/0345492; 2020/0071699.
U.S. Pat. Nos. 4,816,567; 4,895,841; 4,948,807; 5,366,972; 5,482,856; 5,563,142; 5,663,159; 5,811,423; 5,814,639; 5,814,639; 5,905,082; 5,914,331; 5,914,331; 5,922,695; 5,935,946; 5,977,089; 6,043,230; 6,043,230; 6,099,863; 6,238,695; 6,294,540; 6,316,023; 6,335,031; 6,350,736; 6,358,527; 6,417,191; 6,479,284; 6,555,133; 6,627,224; 6,639,071; 6,642,245; 6,677,436; 6,703,396; 6,878,717; 6,939,964; 7,037,917; 7,060,808; 7,160,559; 7,589,078; 7,589,078; 7,727,548; 7,727,552; 7,887,845; 7,906,625; 8,003,789; 8,026,356; 8,039,009; 8,039,009; 8,058,291; 8,168,209; 8,173,708; 8,173,708; 8,183,370; 8,193,165; 8,193,165; 8,283,379; 8,283,379; 8,329,752; 8,329,752; 8,338,486; 8,362,085; 8,362,085; 8,398,980; 8,436,150; 8,460,704; 8,481,554; 8,486,975; 8,592,397; 8,598,233; 8,796,439; 9,126,971; 9,126,971; 9,242,986; 9,296,769; 9,365,647; 9,867,888; 10,253,111.
Wang et al. (2019) MicroRNAs in Alzheimer's disease. Front Genet. 10:153.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method for treating and/or inhibiting progression of a neurological disease, condition, and/or disorder in a subject suffering from and/or at risk for developing neurological disease, condition, and/or disorder, the method comprising administering to the subject a composition comprising a reverse transcriptase inhibitor in an amount and via a route effective for treating and/or inhibiting progression of the neurological disease, condition, and/or disorder in the subject.

2. The method of claim 1, wherein the neurological disease, condition, and/or disorder is selected from the group consisting of Alzheimer's Disease (AD), cerebral amyloid angiopathy, cognitive impairment, mild cognitive impairment, Alzheimer's disease-related attention deficit symptoms, Alzheimer's disease-related neural degeneration, degenerative dementia, senile dementia, cerebral vascular dementia, alcoholic dementia, Parkinson's disease-related dementia, tic disorder, corticobasal ganglionic degeneration, and HIV-associated neurocognitive disorder (HAND), or comprises any combination thereof.

3. The method of claim 1, wherein the composition comprises, consists essentially of, or consists of a reverse transcriptase inhibitor selected from the group consisting of a nucleoside reverse transcriptase inhibitor (NRTI), a non-nucleoside reverse transcriptase inhibitor (NNRTI), or any combination thereof.

4. The method of claim 3, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

5. The method of claim 4, wherein the NNRTI is selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof.

6. The method of claim 1, wherein the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof.

7. The method of claim 1, wherein the composition is formulated for administration in a depot and/or for sustained release.

8. The method of claim 1, wherein the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

9. The method of claim 1, further comprising administering to the subject an additional treatment, optionally an additional AD treatment.

10. The method of claim 9, wherein the additional AD treatment is selected from the group consisting of treatment with an acetylcholinesterase (AChE) inhibitor, optionally donepezil, rivastigmine, and/or galantamine; treatment with an N-methyl-d-aspartate receptor (NMDAR) antagonist, optionally, memantine; treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126, miR-145, miR-195, miR-21, and miR-29b; and combinations thereof.

11. A method for inhibiting development of amyloid beta peptide (Aβ) in a subject suffering from Alzheimer's Disease (AD), the method comprising administering to the subject a composition comprising a reverse transcriptase inhibitor in an amount and via a route effective for treating and/or inhibiting progression of the AD in the subject.

12. The method of claim 11, wherein the composition comprises, consists essentially of, or consists of a reverse transcriptase inhibitor selected from the group consisting of a nucleoside reverse transcriptase inhibitor (NRTI), a non-nucleoside reverse transcriptase inhibitor (NNRTI), or any combination thereof.

13. The method of claim 12, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

14. The method of claim 12, wherein the NNRTI is selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof.

15. The method of claim 11, wherein the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof.

16. The method of claim 11, wherein the composition is formulated for administration in a depot and/or for sustained release.

17. The method of claim 11, wherein the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

18. The method of claim 11, further comprising administering to the subject an additional treatment, optionally an additional AD treatment.

19. The method of claim 18, wherein the additional AD treatment is selected from the group consisting of treatment with an acetylcholinesterase (AChE) inhibitor, optionally donepezil, rivastigmine, and/or galantamine; treatment with an N-methyl-d-aspartate receptor (NMDAR) antagonist, optionally, memantine; treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126, miR-145, miR-195, miR-21, and miR-29b; and combinations thereof.

20. A method for inhibiting microglial cell death, the method comprising administering to a subject in need thereof a composition comprising a reverse transcriptase inhibitor in an amount and via a route effective for inhibiting microglial cell death in the subject.

21. The method of claim 20, wherein the composition comprises, consists essentially of, or consists of a reverse transcriptase inhibitor selected from the group consisting of a nucleoside reverse transcriptase inhibitor (NRTI), a non-nucleoside reverse transcriptase inhibitor (NNRTI), or any combination thereof.

22. The method of claim 21, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

23. The method of claim 21, wherein the NNRTI is selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof.

24. The method of claim 20, wherein the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof.

25. The method of claim 20, wherein the composition is formulated for administration in a depot and/or for sustained release.

26. The method of claim 20, wherein the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

27. The method of claim 20, further comprising administering to the subject an additional treatment.

28. The method of claim 27, wherein the additional treatment is selected from the group consisting of treatment with an acetylcholinesterase (AChE) inhibitor, optionally donepezil, rivastigmine, and/or galantamine; treatment with an N-methyl-d-aspartate receptor (NMDAR) antagonist, optionally, memantine; treatment with a secretase inhibitor, treatment with a beta-site APP-cleaving enzyme (BACE) inhibitor; treatment with an inhibitor of tau aggregation; treatment with an inhibitory nucleic acid, optionally an miRNA, further optionally an miRNA selected from the group consisting of miR-126, miR-145, miR-195, miR-21, and miR-29b; and combinations thereof.

29. A composition for use in treating and/or inhibiting progression of a neurological disease, condition, and/or disorder in a subject suffering from and/or at risk for developing neurological disease, condition, and/or disorder, the composition comprising a reverse transcriptase inhibitor in an amount effective for treating and/or inhibiting progression of the neurological disease, condition, and/or disorder in the subject.

30. A composition for use in inhibiting development of amyloid beta peptide (Aβ) in a subject suffering from Alzheimer's Disease (AD), the composition comprising a reverse transcriptase inhibitor in an amount sufficient to inhibit development of Aβ in the subject.

31. A composition for use in inhibiting microglial cell death, the composition comprising a reverse transcriptase inhibitor in an amount sufficient to inhibit microglial cell death.

32. The composition for use of claim 29, wherein the neurological disease, condition, and/or disorder is selected from the group consisting of Alzheimer's Disease (AD), cerebral amyloid angiopathy, cognitive impairment, mild cognitive impairment, Alzheimer's disease-related attention deficit symptoms, Alzheimer's disease-related neural degeneration, degenerative dementia, senile dementia, cerebral vascular dementia, alcoholic dementia, Parkinson's disease-related dementia, tic disorder, corticobasal ganglionic degeneration, and HIV-associated neurocognitive disorder (HAND), or comprises any combination thereof.

33. The composition for use of claim 29, wherein the reverse transcriptase inhibitor is selected from the group consisting of a nucleoside reverse transcriptase inhibitor (NRTI), a non-nucleoside reverse transcriptase inhibitor (NNRTI), and combinations thereof.

34. The composition for use of claim 33, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof.

35. The composition for use of claim 33, wherein the NNRTI is selected from the group consisting of delavirdine (DLV), efavirenz (EFV), etravirine (ETR), nevirapine (NVP), rilpivirine (TMC278), doravirine (MK-1439), derivatives thereof, pharmaceutically acceptable salts thereof, and combinations thereof.

36. The composition for use of claim 29, wherein the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof.

37. The composition for use of claim 29, wherein the composition is formulated for administration in a depot and/or for sustained release.

38. The composition for use of claim 29, wherein the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

39. The composition for use of claim 29, further comprising an additional active agent.

40. The composition for use of claim 39, wherein the additional active agent is selected from the group consisting of an acetylcholinesterase (AChE) inhibitor, optionally donepezil, rivastigmine, and/or galantamine; an N-methyl-d-aspartate receptor (NMDAR) antagonist, optionally, memantine; a secretase inhibitor, a beta-site APP-cleaving enzyme (BACE) inhibitor; an inhibitor of tau aggregation; an inhibitory nucleic acid, optionally an miRNA, further optionally an miRNA selected from the group consisting of miR-126, miR-145, miR-195, miR-21, and miR-29b; and combinations thereof.

41. A pharmaceutical composition for treating and/or inhibiting progression of neurological diseases, conditions, and/or disorders in a subject in need thereof; and/or for inhibiting development of amyloid beta peptide (Aβ) in a subject in need thereof; and/or for inhibiting microglial cell death, optionally in a subject in need thereof and/or in vitro and/or ex vivo, the pharmaceutical composition comprising, consisting essentially of, or consisting of an effective amount of one or more a reverse transcriptase inhibitors.

42. A composition for preparation of a medicament for treating and/or inhibiting progression of neurological diseases, conditions, and/or disorders in a subject in need thereof; and/or for inhibiting development of amyloid beta peptide (Aβ) in a subject in need thereof; and/or for inhibiting microglial cell death, optionally in a subject in need thereof and/or in vitro and/or ex vivo, the composition comprising, consisting essentially of, or consisting of an effective amount of one or more a reverse transcriptase inhibitors.
